(19) United States
(12) Patent Application Publication
(10) Pub. No.: US 2024/0023881 A1
(43) Pub. Date: Jan. 25, 2024

(54) Medical Diagnostic System

(71) Applicant: KENYATTA CLARKE, BRAMPTON, ON (CA)

(72) Inventor: KENYATTA CLARKE, BRAMPTON, ON (CA)

(21) Appl. No.: 17/870,130

(22) Filed: Jul. 21, 2022

(51) Int. Cl. A61B 5/00

(52) U.S. Cl. CPC A61B 5/441; A61B 5/0064; A61B 5/7246; A61B 5/6898; A61B 2576/02

(57) ABSTRACT

The present invention is medical diagnostic system providing an output scan to the skin surface of a patient and an input of information subsequent to set up a skin we're by the input information is processed and utilized for medical diagnosis.

### BACKGROUND OF THE INVENTION

Advances in technology have led to significant advances in medical diagnostics. Not very long ago, the only definitive way to analyze oxygen levels in human blood must perform in arterial blood gas assay. Today, the pulse oximeter is a very common device that is worn over the fingertip. As is known, the pulse oximeter uses an electronic processor and a pair of small light-emitting diodes (LEDs) facing a photodiode and, when positioned over the fingertip, light absorption readings are correlated to bold oxygen percentages. The relatively simple testing procedure has led to the question, can such measurements be used for a broader application in medical diagnostics? Such a device is needed.

The present invention addresses this need.

### SUMMARY OF THE INVENTION

As generally understood, the present invention provides for medical diagnostic system.

In one embodiment, the invention is a system for medical diagnostics comprising:
a scan processor configured with an output scan device and a return scan sensor, said scan processor configured for scanning the outer skin surface of a patient;
a central processing unit constructing an arranged with memory and input/output, whereby said central processing unit is operatively associated with said scan processor, said memory having patient data acquired from at least one prior scan;
a scan analysis information output from said central processing unit providing information obtained from said outer skin scan.

In one embodiment, the output scan is visible light, UV light, IR light, or combinations thereof.

In one embodiment, the output scan is configured to provide a return scan including at least one of temperature, moisture, number of live skin cells in scan area, number of dead skin cells in scan area, skin surface vibration in reaction to said output scan, or combinations thereof.

In one embodiment, a patient, upon clinical examination who is free of clinical signs of medical illness, ailment, or anomaly, provides an initial scan used as a healthy scan for comparison in subsequent scans.

In one embodiment, the central processing unit memory compares a scan with an initial scan of a specific patient undergoing a scan.

In one embodiment, the central processing unit memory compares a scan with a library of test scans and compares information obtained by said return scan sensor with a known range of scan results stored in said library of test scans.

In one embodiment, the output scan device scans skin of a patient and provides information to said return scan sensor, said information including viral infections, bacterial infections, vaccination compliance, or combinations thereof.

In one embodiment, the scan analysis information output is to a computing device screen, printer, or both.

In one embodiment, the scan analysis information output is to a mobile computing device.

In one embodiment, the scan analysis information output is to a tablet or smartphone.

In one embodiment, the scan analysis information output is to a medical records system maintained by a physician practice, hospital, or other healthcare provider.

In one embodiment, the invention is a system for medical diagnostics consisting of:
a scan processor configured with an output scan device and a return scan sensor, said scan processor configured for scanning the outer skin surface of a patient;
a central processing unit constructing an arranged with memory and input/output, whereby said central processing unit is operatively associated with said scan processor, said memory having patient data acquired from at least one prior scan;
a scan analysis information output from said central processing unit providing information obtained from said outer skin scan, wherein the output scan is visible light, UV light, IR light, or combinations thereof and
the output scan is configured to provide a return scan including at least one of temperature, moisture, number of live skin cells in scan area, number of dead skin cells in scan area, skin surface vibration in reaction to said output scan, or combinations thereof.

In one embodiment, a patient, upon clinical examination who is free of clinical signs of medical illness, ailment, or anomaly, provides an initial scan used as a healthy scan for comparison in subsequent scans.

In one embodiment, the central processing unit memory compares a scan with an initial scan of a specific patient undergoing a scan.

In one embodiment, the central processing unit memory compares a scan with a library of test scans and compares information obtained by said return scan sensor with a known range of scan results stored in said library of test scans.

In one embodiment, the output scan device scans skin of a patient and provides information to said return scan sensor, said information including viral infections, bacterial infections, vaccination compliance, or combinations thereof.

In one embodiment, the scan analysis information output is to a computing device screen, printer, or both.

In one embodiment, the scan analysis information output is to a mobile computing device.

In one embodiment, the scan analysis information output is to a tablet or smartphone.

In one embodiment, the scan analysis information output is to a medical records system maintained by a physician practice, hospital, or other healthcare provider.

### BRIEF DESCRIPTION OF THE SEVERAL VIEWS OF THE DRAWINGS

FIG. 1 is a side perspective view according to one embodiment of the present invention.

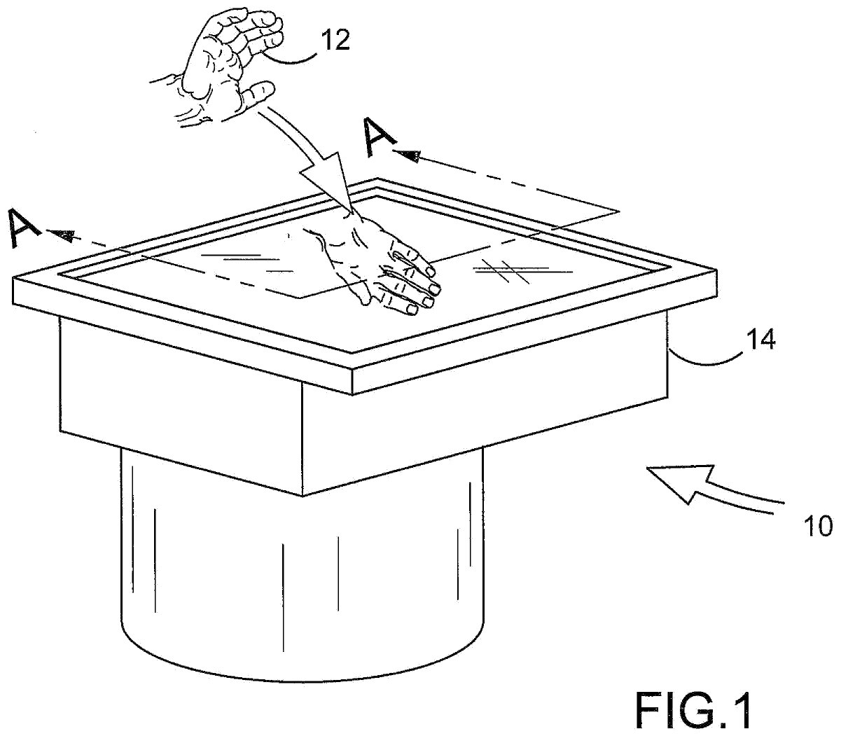

FIG. 2 is a side partial cross section view according to one embodiment of the present invention.

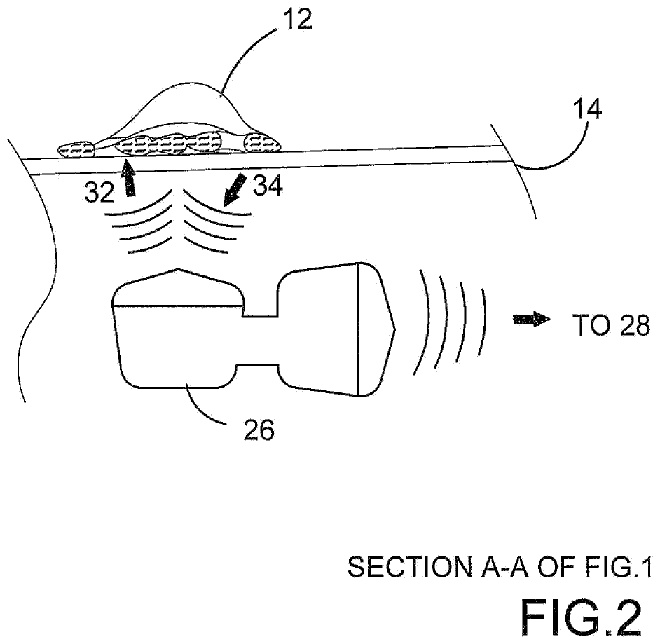

FIG. 3 is a flow chart of the process according to one embodiment of the present invention.

FIG. 4 is a view demonstrating components according to one embodiment of the present invention

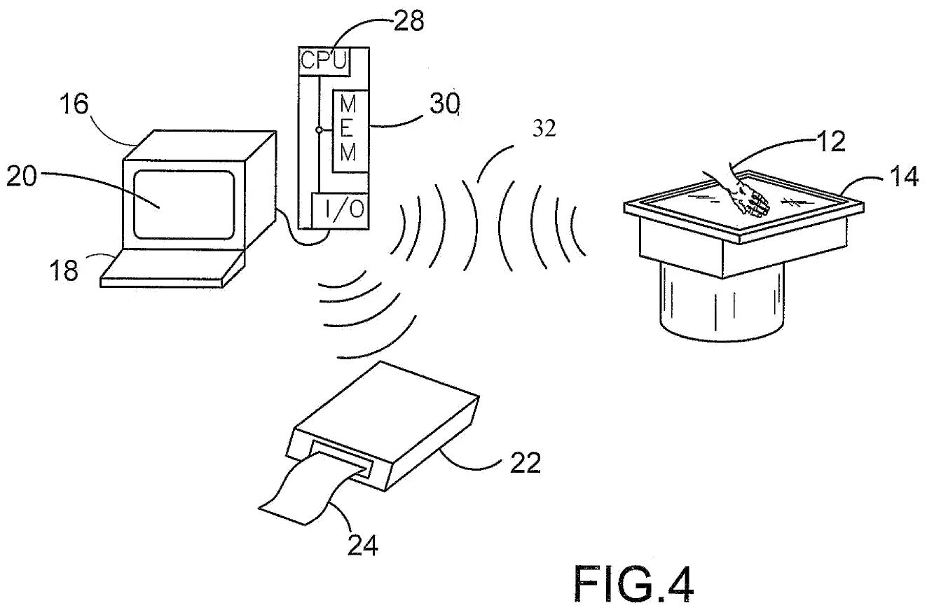

### DETAILED DESCRIPTION OF THE PREFERRED EMBODIMENTS

Although the drawings depict a particularized embodiment utilizing a hand reader, the present invention is contemplated as configurable in any physical structure whereby the inventive scope and concept are achieved.

Similarly, although the figures demonstrate a hand scan, it is contemplated that the present invention is used on any surface skin area where the proper measurements are obtained.

The system 10 will have a reader module 14 constructed and arranged to provide a scan of hand 12. An output scan 32 is directed at hand 12 and a return scan 34 is captured by scan processor 26. The components of scan processor 26 include output scan using a light scan. In one embodiment, the output light scan is visible light, UV light, IR light, or combinations thereof. Scan processor 26 is further configured to capture absorption information in return scan 34. Scan processor 26 is operatively associated with central processing unit (CPU) 28 with is configured with input/output as generally understood and memory 30. Output of scan information is selectively provided on visual screen 20, printer 22 with printed output 24, wireless transmission 32 or combinations thereof. Although the figure demonstrates a conventional desktop computer with keyboard 18 and CPU module 16, the present invention is configurable for use with a tablet, smart phone, or other smart device.

Just as temporal thermometers and oximeters have progressed, the present invention progresses non-invasive medical diagnostics.

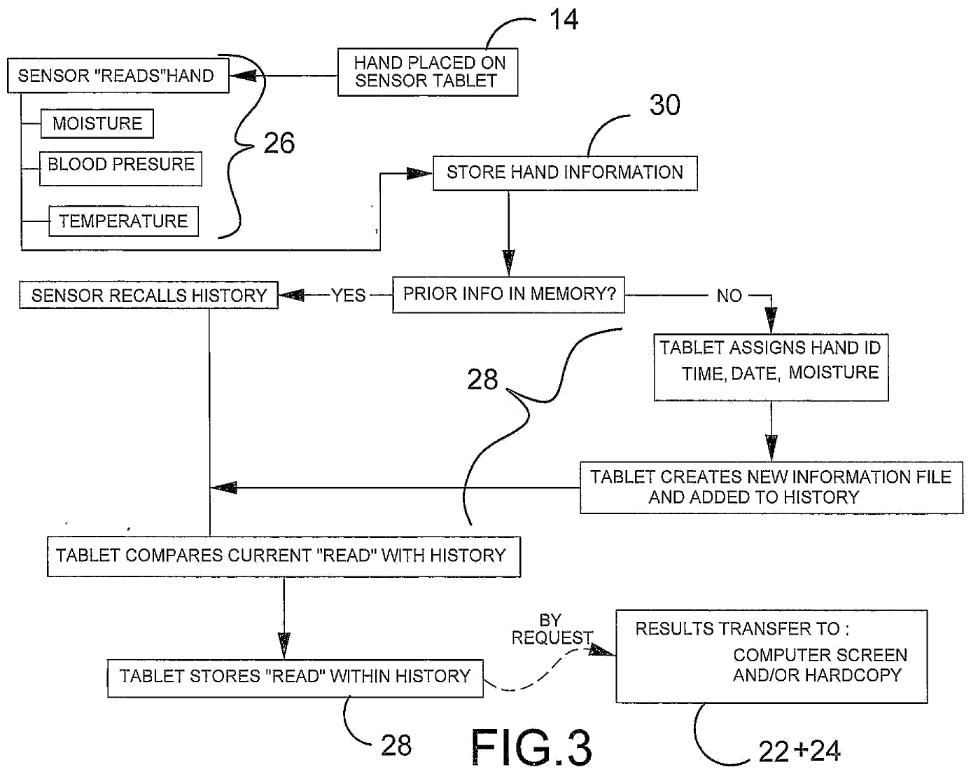

As demonstrated in the flow chart to FIG. 3, the process of the present ivention includes a hand being placed on the sensor and an output sensor being directed towards the skin surface. The system will then provide measurements of:
Skin cells;
Temperature of individual;
Oils in skin; and/or
Dryness of skin.

The system is configured to further be a diagnostic device by altering the sensitivities of vibration, heat, and moisture and alert to medical anomalies.

The system will utilize memory 30 to store significant information relating to normal ranges of healthy persons analyzed with the system of the present invention. The system will then compare a test reading with prior information stored in memory. In one embodiment, the system will provide stored information individualized to a particular patient. In another embodiment, the system will utilize normalized data analysis to provide a range of measurements, where a test reading outside of a recognized range of normal measurements indicate a medical anomaly.

When a particular user or patient has information stored in memory 30, the system will compare current read information with the history. The current read will then be transferred to an output along with analysis.

It is further contemplated that the system of the presentation is configurable to provide information relating to any one or more of viral infections, bacterial infections, vaccination compliance, or combinations thereof.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

What is claimed:

1. A system for medical diagnostics comprising:
a scan processor configured with an output scan device and a return scan sensor, said scan processor configured for scanning the outer skin surface of a patient;
a central processing unit constructing an arranged with memory and input/output, whereby said central processing unit is operatively associated with said scan processor, said memory having patient data acquired from at least one prior scan;
a scan analysis information output from said central processing unit providing information obtained from said outer skin scan.

2. The system of claim 1 wherein said output scan is visible light, UV light, IR light, or combinations thereof.

3. The system of claim 1 wherein said output scan is configured to provide a return scan including at least one of temperature, moisture, number of live skin cells in scan area, number of dead skin cells in scan area, skin surface vibration in reaction to said output scan, or combinations thereof.

4. The system of claim 1 wherein a patient free of clinical signs of medical illness, ailment, or anomaly, provides an initial scan used as a healthy scan for comparison in subsequent scans.

5. The system of claim 1 wherein said central processing unit memory compares a scan with an initial scan of a specific patient undergoing a scan.

6. The system of claim 1 wherein said central processing unit memory compares a scan with a library of test scans and compares information obtained by said return scan sensor with a known range of scan results stored in said library of test scans.

7. The system of claim 1 wherein said output scan device scans skin of a patient and provides information to said return scan sensor, said information including viral infections, bacterial infections, vaccination compliance, or combinations thereof.

8. The system of claim 1 wherein said scan analysis information output is to a computing device screen, printer, or both.

9. The system of claim 1 wherein said scan analysis information output is to a mobile computing device.

10. The system of claim 1 wherein said scan analysis information output is to a tablet or smartphone.

11. The system of claim 1 wherein said scan analysis information output is to a medical records system maintained by a physician practice, hospital, or other healthcare provider.
